(19) United States
(12) Patent Application Publication
(10) Pub. No.: US 2024/0032581 A1
(43) Pub. Date: Feb. 1, 2024

(54) SYNTHETIC NUTRITIONAL COMPOSITIONS TAILORED FOR CHINESE INFANTS

(71) Applicant: SOCIETE DES PRODUITS NESTLE S.A., Vevey (CH)

(72) Inventors: ARISTEA BINIA, Fribourg (CH); IRENE VASSALLO, Lausanne (CH)

(21) Appl. No.: 18/257,330

(22) PCT Filed: Dec. 17, 2021
(86) PCT No.: PCT/EP2021/086483

§ 371 Date: Jun. 14, 2023

(30) Foreign Application Priority Data
Dec. 18, 2020 (EP) 20215301.1

(51) Int. Cl. A23L 33/125; A23L 33/00; A61K 31/702; A61P 3/02

(52) U.S. Cl. CPC A23L 33/125; A23L 33/40; A61K 31/702; A61P 3/02

(57) ABSTRACT

Synthetic nutritional compositions tailored for Chinese infants comprising 3′-SL and 6′-SL in concentrations reflecting those found in human milk produced for infants from the same regional location.

### TECHNICAL FIELD

The invention relates to synthetic nutritional compositions tailored for Chinese infants and to their use to provide administration of an adapted amount of 3′-SL and 6′-SL to a Chinese infant.

### BACKGROUND OF THE INVENTION

Even though breastfeeding is optimal for infants, the existence of certain conditions may mean that it is contraindicated. In such cases, where the sole source of nutrition is not available to infants, alternative strategies to feed them need to be employed. Feeding infants with synthetic nutritional compositions e.g. Infant formula is one such strategy.

The compositions of the aforementioned synthetic nutritional compositions e.g. infant formulas, aim to replicate those of human milk (hereinafter HM). However, replicating HM is not a simple task. HM not only contains numerous components, its composition is extremely dynamic and these dynamic changes remain largely unexplored and uncharacterized.

The inventors have now surprisingly found that the concentration of 3′-SL and 6′-SL in HM may differ depending on regional location.

HMOs are, collectively, the third largest solid constituents in human milk, and a variety of benefits have been associated with them, in consequence, an optimal intake of these compounds in infancy and childhood is believed to be necessary to ensure optimum health and development. HMOs have for example been linked to a variety of biological functions including the establishment of gut microbiota and the promotion of immunity function.

Because such differences in the 3′-SL and 6′-SL concentration of HM have never previously been identified, these differences are not reflected in the compositions of synthetic nutritional compositions available for infants today. Given that HM is considered the gold standard with respect to infant nutrition, there remains a need for synthetic nutritional compositions tailored for infants of specific regions which better reflect these identified differences.

### SUMMARY OF THE INVENTION

The invention is set out in the claims. The inventors have developed synthetic nutritional compositions tailored for Chinese infants comprising 3′-SL and 6′-SL in concentrations that reflect the concentrations of 3′-SL and 6′-SL found in HM produced for infants from the same regional location.

Said synthetic nutritional compositions may, for example, be an infant formula or a composition for infants, for example a supplement, that is intended to be added to or consumed with infant formula.

The 3′-SL and 6′-SL concentration of said synthetic nutritional compositions tailored for Chinese infants may differ. The 3′-SL and 6′-SL concentration may be higher in a synthetic nutritional composition tailored for Chinese infants of age ranging from 0 to 6 months than in a synthetic nutritional compositions tailored for infants of age ranging from 6 to 12 months.

The synthetic nutritional compositions tailored for Chinese infants may be included in a nutritional system.

The 3′-SL and 6′-SL concentrations of the synthetic nutritional compositions of the invention more accurately reflect the 3′-SL and 6′-SL concentrations found in HM produced for Chinese infants, for example of Chinese infants of corresponding or stages of lactation. In light of this and, because HM is considered optimal with respect to infant nutrition, such synthetic nutritional compositions, and the nutritional systems comprising them, can provide an optimized amount of 3′-SL and 6′-SL to a Chinese infant, and may be used to ensure optimum 3′-SL and 6′-SL levels in Chinese infants. The synthetic nutritional compositions may also be used to ensure optimum 3′-SL and 6′-SL intake and levels.

### DETAILED DESCRIPTION

The inventors carried out an analysis of two sialylated HMOs (3-SL and 6′-SL) in Human Breast Milk in relationship to the geographical location of such milk. The contents of 3′ Sialyllactose (3-SL) and 6′ Sialyllactose (6-SL) in maternal milk was compared between the European population and the Chinese population at different lactation stages and results showed a clear difference between Europe and China at each lactation stage tested.

Chinese maternal milk contains statistically significantly lower levels of both 3′SL and 6′SL when compared to the European counterpart.

Based on the findings of the study, the inventors have designed synthetic nutritional compositions tailored for Chinese infants wherein, the 3′-SL and 6′-SL concentration reflects that found in HM produced for a Chinese infant, for example for a Chinese infant of the same age (at the corresponding lactation stage).

### Definitions

Within the context of the present invention, the term “Chinese infant” identifies an infant born in the Chinese region and of Chinese ethnicity. In one embodiment, the Chinese infant is born from a mother of Chinese ethnicity.

The term “synthetic nutritional composition tailored for a Chinese infant” as used herein refers to any synthetic nutritional composition that is intended to be consumed by a Chinese infant and that is specifically adapted to the nutritional needs of said infant.

The term “synthetic nutritional composition tailored for a Chinese infant of a specific age” as used herein refers to any synthetic nutritional composition that is intended to be consumed by a Chinese infant as above defined of a specific age and, that is specifically adapted to the nutritional needs of said infant at the specific age.

Non limiting examples of synthetic nutritional compositions tailored for a Chinese infant include; infant formulae, a composition for infants that is intended to be added to or consumed with Infant formula, e.g. a supplement or fortifier, and food stuffs intended for consumption by infants either alone or in combination with HM or Infant formula e.g. complementary foods.

The term “infant” as used herein refers to a human infant of 12 months of age or less.

In one embodiment, the infant according to the present invention is in need of 3′-SL and 6-SL.

In another embodiment, the infant is in need of 3′-SL and 6′-SL and has non-optimal 3′-SL and 6′-SL levels' intake.

In an embodiment of the present invention there is provided a synthetic nutritional composition tailored for a Chinese, for example a Chinese infant of a specific age, comprising 3′-SL and 6′-SL in a concentration reflecting that found in human milk produced for a Chinese infant, for example a Chinese infant of the same age (at the corresponding lactation age).

In one embodiment, the synthetic nutritional composition tailored for a Chinese infant, for example a Chinese infant of a specific age, comprises, after reconstitution, a concentration of 3′-SL and 6′-SL reflecting that found in human milk produced for a Chinese infant, for example a Chinese infant of the same age (at the corresponding lactation age).

In an embodiment of the present invention said synthetic nutritional composition is tailored for a Chinese infant of a specific age selected from the group consisting of from 0 to 6 months of age and from 6 to 12 months of age.

In an embodiment the synthetic nutritional composition is tailored for an infant of age ranging from 0 to 6 months the 3′-SL concentration ranges from 40 to 100 mg/L, for example 70 to 90 mg/L.

In an embodiment the synthetic nutritional composition is tailored for an infant of age ranging from 6 to 12 months the 3′-SL concentration ranges from 40 to 100 mg/L, for example 70 to 90 mg/L.

In an embodiment the synthetic nutritional composition is tailored for an infant of age ranging from 0 to 6 months the 6′-SL concentration ranges from 70 to 220 mg/L, for example 120 to 180 mg/L.

In an embodiment the synthetic nutritional composition is tailored for an infant of age ranging from 6 to 12 months the 6′-SL concentration ranges from 25 to 60 mg/L, for example 30 to 50 mg/L.

The 3′-SL and 6′-SL concentration of the synthetic nutritional compositions tailored for a Chinese infant, for example a Chinese infant of a specific age, as defined herein is expressed in mg/ml. This typically refers to the 3′-SL and 6′-SL concentration of a reconstituted synthetic nutritional compositions tailored for a Chinese infant, for example a Chinese infant of a specific age. In one embodiment of the present invention, the synthetic nutritional composition is an Infant formula which is reconstituted in water.

The term “3′-SL” refers to the oligosaccharide 3′-sialyllactose, having the structure:

α-Neu5Ac(2→3)-β-D-Gal(1→4)-D-Glc

The term “6′-SL” refers to the oligosaccharide 3′-sialyllactose, having the structure:

α-Neu5Ac-(2→6)-β-D-Gal-(1→4)-D-Glc

The 3′-SL and 6′-SL concentration of a composition can be measured by methods well known in the art. For example, the 3′-SL and/or 6′-SL concentration of HM or a synthetic nutritional composition of the invention may be measured by liquid chromatography. A method for the measurement of 3′-SL and 6′-SL in HM or in the synthetic nutritional composition as disclosed herein is set out in the examples included herein.

Any form of 3′-SL and/or 6′-SL is suitable for administration to an infant to whom the synthetic nutritional composition is directed may be comprised within in the synthetic nutritional compositions of the invention.

The synthetic nutritional compositions tailored for a Chinese infant can also comprise any other ingredients or excipients known to be employed in the type of synthetic nutritional composition in question e.g. infant formula, a composition for infants that is intended to be added to or consumed with Infant formula, or food stuffs intended for consumption by infants e.g. complementary foods.

In an embodiment of the present invention the synthetic nutritional compositions tailored for a Chinese infant of a specific age are selected from the group consisting of: infant formula, and a composition for infants that is intended to be added to or consumed with Infant Formula.

Non-limiting examples of ingredients known to be employed in the typed of synthetic nutritional compositions in question include: proteins, amino acids, carbohydrates, oligosaccharides, lipids, prebiotics or probiotics, essential fatty acids, nucleotides, nucleosides, other vitamins, minerals and other micronutrients.

Non limiting examples of proteins include: casein, alpha-lactalbumin, whey, soy protein, rice protein, corn protein, oat protein, barley protein, wheat protein, rye protein, pea protein, egg protein, sunflower seed protein, potato protein, fish protein, meat protein, lactoferrin, serum albumin, immunoglobins, and combinations thereof.

Non-limiting examples of amino acids include leucine, threonine, tyrosine, Isoleucine, arginine, alanine, histidine, isoleucine, proline, valine, cysteine, glutamine, glutamic acid, glycine, serine, arginine, lysine, methionine, phenylalanine, tryptophane, asparagine, aspartic acid, and combinations thereof.

Non-limiting examples of carbohydrates include lactose, saccharose, maltodexirin, starch, and combinations thereof.

Non-limiting examples of lipids include: palm olein, high oleic sunflower oil, high oleic safflower oil, canola oil, fish oil, coconut oil, bovine milk fat, and combinations thereof.

Non-limiting examples of essential fatty acids include: linoleic acid (LA), α-linolenic acid (ALA) and polyunsaturated fatty acids (PUFAs). The nutritional compositions of the invention may further contain gangliosides monosialoganglioside-3 (GM3) and disialogangliosides 3 (GD3), phospholipids such as sphingomyelin, phospholipids phosphatidylcholine, phosphatidylethanolamine, phosphatidylinositol, phosphatidylserine, and combinations thereof.

None limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

Further examples of oligosaccharide are described in Wrodnigg, T. M.; Stutz, A. E. (1999) Angew. Chem. Int. Ed. 38:827-828 and in WO 2012/069416

Non limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof.

Non-limiting examples of Nucleotides include: cytidine monophosphate (CMP), uridine monophosphate (UMP), adenosine monophosphate (AMP), guanosine monophosphate (GMP), and combinations thereof.

Non limiting examples of other vitamins and minerals include: vitamin B1, vitamin B2, vitamin B6, vitamin B12, vitamin E. vitamin K. vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, L-carnitine, and combinations thereof. Minerals are usually added in salt form.

Other suitable and desirable ingredients of synthetic nutritional compositions, that may be employed in the synthetic nutritional compositions tailored for Chinese infants, for example Chinese infants of specific ages, are described in guidelines issued by the Codex Alimentarius with respect to the type of synthetic nutritional composition in question e.g. Infant formula, Infant formula fortifier, supplements or food stuffs intended for consumption by infants e.g. complementary foods.

The synthetic nutritional compositions tailored for Chinese infants may be prepared by methods well known in the art for preparing the type of synthetic nutritional composition in question e.g. infant formulae, a composition for infants that is intended to be added or diluted with Infant Formula e.g. IF fortifier, or food stuffs intended for consumption by infants either alone or in combination with HM or infant formula e.g. complementary foods.

An exemplary method for preparing a synthetic nutritional compositions tailored for Chinese infants that is powdered infant formula is as follows. A protein source, carbohydrate source (including 3′-SL and 6′-SL), and fat source may be blended together in appropriate proportions. Emulsifiers maybe included in the blend. Vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger.

The liquid mixture may then be cooled to about 60° C. to about 85° C.; for example by flash cooling. The liquid mixture may then be homogenised; for example in two stages at about 7 MPa to about 40 MPa in the first stage and about 2 MPa to about 14 MPa in the second stage. The homogenised mixture may then be further cooled to add any heat sensitive components such as vitamins and minerals (if not added earlier, lutein may be added at this stage, for example as part of a vitamin premix). The pH and solids content of the homogenised mixture is conveniently standardised at this point.

The homogenised mixture can be transferred to a suitable drying apparatus such as a spray drier or freeze drier and converted to powder. The powder should have a moisture content of less than about 3% by weight.

If it is desired probiotic(s) may be added, they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the age tailored powdered infant formula by dry mixing.

The synthetic nutritional compositions tailored for a Chinese infant of a specific age may also be prepared from a non-region tailored synthetic nutritional composition.

In one embodiment, the synthetic nutritional compositions of the invention may be prepared from a non-regional tailored synthetic nutritional composition by measuring out an appropriate amount of said non-regional tailored synthetic nutritional composition and mixing it with an additive and/or diluent e.g. 3′-SL and 6′-SL and/or water.

In an aspect of the invention there is a provided method of preparing a synthetic nutritional composition tailored for a Chinese infant comprising of: measuring out an appropriate amount of a non-region tailored synthetic nutritional composition and mixing it with an additive and/or a diluent e.g. water so as to arrive at a synthetic nutritional composition tailored to a Chinese infant in accordance with the invention.

In one embodiment, the additive comprises of 3′-SL and/or 6′-SL.

The additive may be a tailored additive comprising 3′-SL and/or 6′-SL in a particular concentration so that when mixed with the non-region specific synthetic nutritional composition, and optionally a diluent, the resulting mixture is a synthetic nutritional composition tailored to a Chinese infant in accordance with the invention.

The non-region specific synthetic nutritional composition can be prepared by methods well known in the art for the type of composition in question e.g. as laid out above for infant formula.

One or more of the synthetic nutritional compositions tailored for a Chinese infant of a specific age can be included in a nutritional system.

The term “nutritional system” as used herein refers to a collection of more than one synthetic nutritional composition advertised or sold as part of the same product range e.g. a collection of infant formulas sold under the same brand and adapted to the nutritional needs of infants of different regions, differing ages and/or genders and/or delivered by different methods e.g. C-section. In one embodiment, the synthetic nutritional compositions making up the nutritional system are packaged individually e.g. in capsules or boxes. Said packages can be sold individually, grouped together e.g. wrapped by plastic film or combined in a box, or in a combination of these two ways. The nutritional system may also comprise synthetic nutritional compositions for children older than 12 months.

In a further aspect of the present invention there is provided a nutritional system comprising a synthetic nutritional composition tailored for a Chinese infant, for example a Chinese infant of a specific age, in accordance with the invention.

In an embodiment the nutritional system comprises a synthetic nutritional composition tailored for a Chinese infant of age ranging from 0 to 6 months, as disclosed herein, and a synthetic nutritional composition tailored for a Chinese infant age ranging from 6 to 12 months, as disclosed herein, wherein, the 3′-SL and/or 6′-SL concentration of said synthetic nutritional composition tailored for a Chinese infant of age ranging from 0 to 6 months is higher than that of said synthetic nutritional composition tailored for a Chinese infant age ranging from 6 to 12 months.

Because HM is the gold standard when it comes to infant nutrition, and because the 3′-SL and/or 6′-SL concentration of the synthetic nutritional compositions of the invention better reflect the 3′-SL and/or 6′-SL concentrations found in HM expressed for Chinese infants they, and the nutritional systems comprising them, may be used to provide an optimum amount of 3′-SL and/or 6′-SL to a Chinese infant and to ensure optimum 3′-SL and/or 6′-SL levels.

3′SL and 6′SL are major human milk sialyl-oligosaccharides shown to be linked with benefits for brain and bone development (https://pubmed.ncbi.nlm.nih.gov/31746283/, https://pubmed.ncbi.nlm.nih.gov/31138692/31138692). Metabolic health outcomes might also be improved by 3′SL intervention (https://pubmed.ncbi.nlm.nih.gov/32694823/).

In another aspect of the present invention there is provided a specific synthetic nutritional composition of the invention and/or a nutritional system of the invention for use to prevent and/or treat non-optimal 3′-SL and/or 6′-SL levels in a Chinese infant, for example an infant of age ranging from 0 to 6 months, or an infant of age ranging from 6 to 12 months.

The nutritional system may provide an optimum amount of 3′-SL and/or 6′-SL to a Chinese infant up to 12, 11, 10, 9, 8, 7, 6, 5, 4, 3, 2, 1 months of age.

In another aspect of the present invention there is provided a method treating and/or preventing non-optimal 3′-SL and/or 6′-SL levels in a Chinese infant or for providing an optimum amount of 3′-SL and or 6′-SL to an infant comprising:
a) Optionally preparing a synthetic nutritional composition, tailored for a Chinese infant from an non-region tailored synthetic nutritional composition;
b) Feeding a synthetic nutritional composition tailored for a Chinese infant to such infant.

As stated herein, a synthetic nutritional composition tailored for a Chinese infant, according to the invention, may be prepared from a non-region tailored synthetic nutritional composition. Accordingly, in another aspect of the present invention there is provided a kit for providing an optimized amount of 3′-SL and/or 6′-SL to a Chinese infant comprising:
a) A non-region tailored synthetic nutritional composition
b) A label indicating dosage requirements for said non-region tailored synthetic nutritional composition so as to arrive at a synthetic nutritional composition tailored for a Chinese infant in accordance with the invention.

The dosage requirements may be with respect to the quantity of the non-region tailored synthetic nutritional composition employed and/or the consumption frequency e.g. 4 times per day.

The kit and method may provide an optimized amount of 3′-SL and/or 6′-SL to a Chinese infant, in particular a Chinese infant age ranging from 0 to 6 months, or an infant age ranging from 6 to 12 months.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

There now follows a series of non-limiting examples that serve to illustrate the invention.

### EXAMPLES

### Example 1

Comparison of Content of 3′-SL and 6′-SL in Human Breast Milk from Different Regions

Data from human breast milk samples from five different studies were used by the inventors for a statistical analysis targeted to compare the 3′-SL and 6′-SL content in HBM coming from different regions:
Study 2: Longitudinal Study in Europe (follow up from birth until 4 months)
Study 5: Cross-sectional Study in China (follow up from birth until 8 months)
Study 6: Longitudinal Study in Germany (follow up from 3 months until 12 months)
Study 8: Longitudinal Study in Ireland (follow up from birth until 10 weeks)
Study 9: Longitudinal Study in Switzerland (follow up from birth until 8 weeks)

Only term delivery were included in the analysis (between 37 and 42 weeks of gestation age). Table 1 below presents a summary of the key elements of the studies used in the analysis, while in the following paragraphs a more detailed description of the clinical study follows:

TABLE 1
Analysis population - summary statistics

| | STUDY 2 | STUDY 5 | STUDY 6 | STUDY 8 | STUDY 9 |
|---|---|---|---|---|---|
| Study Design | Longitudinal | Cross-sectional | Longitudinal | Longitudinal | Longitudinal |
| Duration of Followup | 4 months | 8 months | 12 months | 4 months | 4 months |
| Population | European | Chinese | German | Irish | Swiss |
| Sample Size for the | 303 | 86-90 | 156 | 24 | 28 |
| analysis | | (depending on the | | | |
| | | lactation stage) | | | |

1.1 Description of the Studies

Study 2

Study Population:

Study 2 is a multicenter, longitudinal, observational, exploratory cohort study designed to characterize HM and its association with maternal and infant parameters. HM as well as multiple maternal and infant parameters were collected postpartum at 6 visits (V) (V1, 0-3 days; V2, 17±3 days; V3, 30±3 days; V4, 60±5 days; V5, 90±5 days; V6, 120±5 days). Enrollment was performed at multiple sites in 7 European countries, including Spain, France, Italy, Norway, Portugal, Romania, and Sweden. The total duration of participation was 4 months after infant birth. Trained and certified research nurses and assistants collected all data. All data captured were directly entered into a secured web-based database (Medidata Rave edc 5.6.4). The procedures followed were in accordance with the ethical standards of the respective local ethical committees in each country.

Analysis Population:

A total of 370 participants from 7 European countries. Participants were counted as pairs of mothers and infants. The analysis was performed on 330 participants (pairs) after removal of pairs who did not satisfy the inclusion-exclusion criteria and with incomplete information on HM composition. Number of subject by country:
ESP=9
FRA=87
ITA=16
NOR=9
PRT=94
ROU=41
SWE=40

Study 5

Study Population:

This is an observational, cross sectional, multi-center study aiming at evaluating nutrients composition of breast milk and nutrition intake of Chinese lactating mothers in 3 cities of China (Beijing, Suzhou and Guangzhou). In Beijing, Suzhou and Guangzhou there were 220, 180 and 180 of healthy lactating mothers respectively, enrolled at different lactation stages (within 5-11 days, 12-30 days, 31-60 days, 61-120 days and 121-240 days postpartum). In total, 580 mothers were enrolled.

Analysis Population:

580 mothers were enrolled in the study. The analysis was performed on a subset of 443 mothers (and their child) due to incomplete information on HM composition.

Study 6

This is a large population-based longitudinal cohort study conducted in Germany. The cohort is a sample of 1,000 pregnant women and their offspring. Prenatal examinations are conducted during the 24th to 26th and 34th to 36th week of gestation. During the first year of life, data is collected from children at 3, 6 and 12 month of age.

Analysis Population:

Of the enrolled mothers, 156 had complete HM information. They will be included in the analysis.

Study 8

Is a longitudinal cohort study conducted in Ireland. The study enrolled 70 mothers who gave birth to full-term (>37 weeks gestation, FT), pre-term (32-37 weeks, PT) and very pre-term (<32 weeks, VPT) infants. FT milk was collected at 1, 3, 6 and 10 weeks post-partum (PP), while PT and VPT milk was collected weekly until the FT due date of the infant and subsequently followed the FT protocol.

Analysis Population:

This analysis focused on mothers delivering term infants. Hence, out of the 70 enrolled mothers, the 24 mothers that had complete information on HM composition from the term group were considered in this analysis.

Study 9

Study Population:

Open, single-centre, exploratory study on healthy lactating mothers delivering term (gestational age (ga) between 37 and not above 42 weeks) and preterm (ga between 28 and 32 weeks) infants.

A total of 61 mothers (34 in the term group and 25 in the preterm group, 6 mothers in the latter group delivered twins) were enrolled in the study. For subjects who delivered pre-term babies, milk samples were collected once a week until discharge from hospital and then every 2 weeks until 8 weeks after birth (at most 12 visits post-screening). For subjects who delivered term babies, milk samples were collected once a week until 8 weeks after birth (8 visits post-screening).

Analysis Population:

This analysis focused on mothers delivering term infants. Hence, out of the 61 enrolled mothers, the 28 mothers that had complete information on HM composition from the term group were considered in this analysis.

1.2 Measurement of 3′-SL and 6′-SL Concentrations in Samples:
3′SL and 6′SL concentrations were determined by liquid chromatography with fluorescence detection after labelling the oligosaccharides with 2-aminobenzamide, as described previously (Austin & Benet, 2018, Analytica Chimica Acta, 1010: 86-96; Austin, et al., 2016, Nutrients, 8: 346).

1.3 Statistical Method

3′SL and 6′SL concentrations were averaged by lactation stages using the following thresholds:
STAGE 1: from 12 days to 4 Months
STAGE 2: from >than 4 Months up to 8 Months

To assign each sample to a lactation stage we used the exact day of lactation from birth. In Study 5 only the visit windows are available (example 5-11 days) and to assign to a specific lactation stage the median day of the visit window was used. Mature Milk was limited up to 8 months to be able to compare between China and Europe (in china the follow-up was up to 8 months). Unfortunately, there is no colostrum in the Chinese study.

Three analyses have been performed:

1) Test for difference in 6′SL and 3′SL concentration between China and Europe by pulling together all European studies. At each lactation stage a t-test comparing Europe and China HMOs concentrations was performed.

2) Test for difference in 6′SL and 3′SL concentration between China and Europe Studies keeping all the included studies separated. An Anova followed by Tukey post-hoc correction was performed at each lactation stages comparing the HMOs concentration between each included study.

1.4 Results

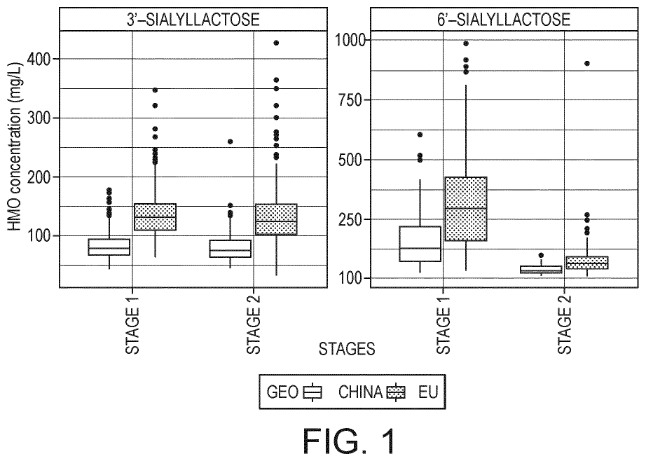

The statistical analysis shows a clear difference between the level of 3′SL and 6′SL in China and Europe. At each lactation stage tested, China shows a reduced level of both HMOs tested. Table 2 shows the results of the statistical test performed (t-test) which are also represented graphically in FIG. 1.

TABLE 2
Comparison of concentration (in mg/L) of 3′SL (X3SL) and
6′SL (X6SL) in milk from Chinese or European mothers

| STAGES | Mean_China | Mean_EU | p. value |
|---|---|---|---|
| X3SL | | | |
| STAGE 1 | 84.11566 | 136.08918 | 0** |
| STAGE 2 | 82.55611 | 133.11811 | 0** |
| X3SL | | | |
| STAGE 1 | 155.58629 | 309.39576 | 0** |
| STAGE 2 | 38.80444 | 75.65433 | 0** |

^{a} Welch Two Sample t-test

Based on such results, the present inventors have determined for 3′-SL and 6′-SL amounts which result to be particularly adapted to Chinese infants as reflecting the content of such Human milk oligosaccharides in human breast milk of the mothers of Chinese infants.

Such amounts are reported in table 3 below for infants between 0 and 6 months of age (thus consuming Stage 1 Infant Formulas) and infants between 6 and 12 months of age (thus consuming Stage 2 Infant Formulas).

TABLE 3

| | Amount of 3′SL (mg/L) | Amount of 6′SL (mg/L) |
|---|---|---|
| Stage 1 | 40-100 | 70-220 |
| Stage 2 | 40-100 | 25-60 |

### Example 2

Examples of Synthetic Nutritional Compositions (Infant Formulas) Tailored to Chinese Infants of Specific Ages are Given in Table 5

TABLE 5

| | up to 6 | from 6 to 12 |
|---|---|---|
| | months of age | months of age |
| Ingredients | Per Litre | Per Litre |
| Energy (kcal) | 670 | 670 |
| Protein (g) | 12.1 | 12.1 |
| Fat (g) | 35.639 | 35.649 |
| Cholesterol (g) | 0.061 | 0.051 |
| Linoleic acid (g) | 5.3 | 5.3 |
| α-Linolenic acid (mg) | 675 | 675 |
| Lactose (g) | 74.7 | 74.7 |
| Prebiotic (100% GOS) (g) | 4.3 | 4.3 |
| Minerals (g) | 2.5 | 2.5 |
| Na (mg) | 150 | 150 |
| K (mg) | 590 | 590 |
| Cl (mg) | 430 | 430 |
| Ca (mg) | 410 | 410 |
| P (mg) | 210 | 210 |
| Mg (mg) | 50 | 50 |
| Mn (μg) | 50 | 50 |
| Se (μg) | 13 | 13 |
| Lutein (μg) | 61 | 48 |
| Vitamin D (μg) | 10 | 10 |
| Vitamin E (mg TE) | 5.4 | 5.4 |
| Vitamin K1 (ug) | 54 | 54 |
| Vitamin C (mg) | 67 | 67 |
| Vitamin B1 (mg) | 0.47 | 0.47 |
| Vitamin B2 (mg) | 1 | 1 |
| Niacin (mg) | 6.7 | 6.7 |
| Vitamin B6 (mg) | 0.5 | 0.5 |
| Lactoferrin (bovine) g | 1 | 1 |
| Folic acid (μg) | 60 | 60 |
| Pantothenic acid (mg) | 3 | 3 |
| Vitamin B12 (μg) | 2 | 2 |
| Biotin (μg) | 15 | 15 |
| Choline (mg) | 67 | 67 |
| Fe (mg) | 8 | 8 |
| I (μg) | 100 | 100 |
| Cu (mg) | 0.4 | 0.4 |
| Zn (mg) | 5 | 5 |
| 3-SL | 85 | 80 |
| 6-SL | 155 | 40 |

1. A synthetic nutritional composition tailored for a Chinese infant comprising 3′-SL and 6′-SL in a concentration reflecting that found in human milk produced for an infant from the same regional location.

2. A synthetic nutritional composition according to claim 1 wherein said synthetic nutritional composition is tailored for a Chinese infant of a specific age selected from the group consisting of 0 to 6 months of age, and from 6 to 12 months of age.

3. A synthetic nutritional composition according to claim 1 wherein the synthetic nutritional composition has a concentration of 3′-SL ranging from 40 to 100 mg/L and a concentration of 6′-SL is ranging from 25 to 220 mg/L after reconstitution of the product.

4. A synthetic nutritional composition tailored for a Chinese infant according to claim 1 which is an infant formula or a supplement which is added to or admixed with infant formula.

5. A synthetic nutritional composition tailored for a Chinese infant according to claim 1 wherein the concentration of 3′-SL is from 40 to 100 mg/L and the concentration of 6′-SL is from 70 to 220 mg/L after reconstitution of the product.

6. A method for providing nutrition to a Chinese infant between 0 and 6 months of age comprising administering a synthetic nutritional composition tailored for the infant and having a concentration of 3′-SL ranging from 40 to 100 mg/L and a concentration of 6′-SL ranging from 25 to 60 mg/L after reconstitution of the product.

7. (canceled)

8. A method according to claim 6 for use in ensuring administration of optimal levels of 3′-SL and 6′-SL in a Chinese infant.

9. A method according to claim 6 for use in promoting brain and/or bone development and/or promoting metabolic health.

10-14. (canceled)
